(19) United States
(12) Patent Application Publication
(10) Pub. No.: US 2022/0126017 A1
(43) Pub. Date: Apr. 28, 2022

(54) BODY CONFORMING WEARABLE DEVICE FOR PROVIDING OUTPUT AND INPUT FOR A DRUG DELIVERY SYSTEM

(71) Applicant: Insulet Corporation, Acton, MA (US)

(72) Inventor: Nicholas CONTE, Harvard, MA (US)

(21) Appl. No.: 17/506,145

(22) Filed: Oct. 20, 2021

Related U.S. Application Data
(60) Provisional application No. 63/104,740, filed on Oct. 23, 2020.

(51) Int. Cl. A61M 5/142; A61M 5/172; A61B 5/00

(52) U.S. Cl. CPC A61M 5/14244; A61M 5/1723; A61B 5/742; A61M 2005/14208; A61B 5/6824; A61B 5/6825; A61B 5/6835

(57) ABSTRACT

Exemplary embodiments provide a management wearable device with a body conforming housing. The wearable device may be worn on parts of the user's body, such as the hand, wrist, arm or leg. This eliminates the need for the user to hand carry a device. The wearable device may include a display for displaying information to the user. Where the drug delivery system delivers insulin, the display may be used to display information such as blood glucose concentration readings, insulin on board and other information. The wearable device may also include input elements, such as push buttons, knobs, keys or switches, for providing information, entering commands or initiating or halting activity.

### CROSS-REFERENCE TO RELATED APPLICATIONS

This application claims the benefit to U.S. Provisional Application No. 63/104,740, filed Oct. 23, 2020, the entire contents of which are incorporated herein by reference in their entirety.

### BACKGROUND

Drug delivery systems are available in different configurations. For example, drug delivery systems may include a drug pump for delivering drugs to a user. The drug pump typically has a control mechanism that controls delivery of the drug to a user in accordance with a control algorithm. The drug pump may, in some cases, be wearable on the user. In some uses of a drug delivery system, such as with insulin delivery, a dedicated handheld manager device may be provided for displaying information to a user regarding the drug delivery and for providing control of the drug pump.

### SUMMARY

In accordance with an exemplary embodiment, a wearable device includes a user interface for displaying information to a user. The wearable device also includes one or more activatable input elements formed in the wearable device for enabling the user to provide input to a drug delivery device. The wearable device further includes a communication interface for communicating with one or more external devices and a body conforming housing configured to make the wearable device wearable by the user.

The body conforming housing of the wearable device may include at least one elastomeric layer. The user interface may include a display. The user interface may be configured to display user blood glucose information from a glucose monitor. The communication interface may enable communication with the glucose monitor. The user interface of the wearable device may be configured to display information regarding a quantity of insulin on board (JOB). The communication interface may enable communication with the drug delivery pump. The communication interface may be a wireless communication interface. The one or more activatable input elements formed in the wearable device may be one of a button, a switch, a key, a wheel, a slider (such as a capacitive sensor that a user can slide their finger along) or a knob. The activatable input elements may include an activatable input structure for requesting delivery of a bolus of insulin to the user. The wearable device may be structured to be worn on at least one of an arm of the user, a wrist of the user or a hand of the user. The drug delivery pump may be an insulin delivery pump.

In accordance with an exemplary embodiment, a drug delivery system includes a drug delivery pump assembly. The drug delivery pump assembly includes a source of a drug to be delivered and a pump for pumping the drug from the source. The drug delivery pump assembly includes a user interface for interfacing the pump with the user for delivery of the drug to the user and also includes a drug delivery pump assembly housing for housing the drug delivery pump assembly and configured to be worn by the user. The drug delivery system additionally includes a wearable device having a user interface for displaying information to the user. The wearable device has one or more activatable input elements for enabling a user to provide input to the drug delivery pump assembly. The wearable device further includes a body conforming housing configured to make the wearable device wearable by the user.

The drug may be insulin, and the source of the drug may be an insulin reservoir. The body conforming housing of the wearable device may include at least one elastomeric layer. The user interface of the wearable device may be configured to display user blood glucose information. The user interface of the wearable device may be configured to display information regarding a quantity of insulin in the source. The user interface of the wearable device may be configured to display information regarding a quantity of insulin on board for the user. The one or more activatable input elements formed in the wearable device may be one of a button, a switch, a key, a wheel, a slider or a knob. The wearable device may be structured to be worn on at least one of an arm of the user, a wrist of the user or a hand of the user.

### BRIEF DESCRIPTION OF THE DRAWINGS

FIG. 1A depicts an illustrative drug delivery system for an exemplary embodiment.

FIG. 1B depicts components of the drug delivery system positioned relative to a user in an illustrative arrangement.

FIG. 2 depicts portions of the illustrative drug delivery system in more detail.

FIG. 3 depicts more detail of a wearable management device that is part of the drug delivery system in an exemplary embodiment.

FIG. 4A depicts a wearable management device configured to be worn on a hand of a user.

FIG. 4B depicts a wearable management device configured to be worn on a wrist, an arm or a leg of a user.

FIG. 4C depicts a wearable management device in an unrolled configuration.

FIG. 4D depicts a wearable management device in a rolled configuration.

FIG. 5A depicts an instance in which a wearable management device includes an elastomer layer and an electrical circuitry layer.

FIG. 5B depicts an instance in which a wearable management device includes two elastomer layers and an electrical circuitry layer.

FIG. 6 depicts a diagram of illustrative commands/actions that may be initiated from the wearable management device.

FIG. 7A depicts a flowchart of illustrative steps that may be performed to obtain an insulin on board (JOB) value for a user.

FIG. 7B shows an example of a wearable management device where the IOB for a user is obtained.

FIG. 8A depicts a flowchart of illustrative steps that may be performed to obtain a blood glucose concentration value for a user.

FIG. 8B depicts an example of a wearable management device where the blood glucose concentration value for a user is obtained.

FIG. 9A depicts a flowchart of illustrative steps that may be performed to request a drug bolus.

FIG. 9B depicts an example of a wearable management device where the user requests a drug bolus.

FIG. 10A depicts a flowchart of illustrative steps that may be performed to request an adjustment in a basal delivery amount.

FIG. 10B depicts an example of a wearable management device where the user requests an adjustment to the basal delivery amount.

### DETAILED DESCRIPTION

One of the difficulties with conventional drug delivery systems is that they require at least one handheld device. For instance, many conventional drug delivery systems require a dedicated handheld manager device for managing the drug delivery system. In such conventional drug delivery systems, the user is responsible for ensuring that the dedicated handheld manager device remains in proximity to the user. Unfortunately, the user may forget the device and as a result, may not have access to the dedicated handheld manager device. In such a case, a user may not be able to access the functionality provided by the dedicated handheld manager device. In addition, keeping track of the device and remembering to carry the device as the user moves about his/her day may be burdensome.

Other conventional drug delivery systems may rely upon an application installed on a mobile phone to provide the functionality provided by dedicated handheld manager device. Use of such applications also has drawbacks. The user is responsible for carrying the mobile phone on which the application is installed and remembering to bring the mobile phone with him/her as he/she moves about. Since a mobile phone is used so frequently by many users in their daily lives, the mobile phone may run out of charge, may get broken, may get lost or may become corrupted by a virus. Such events may prevent or limit use of the application.

Exemplary embodiments overcome these limitations of conventional drug delivery systems by providing a manager device that is wearable. The manager device has a body conforming housing. The manager device may be worn on parts of the user's body, such as the hand, wrist, arm or leg. This eliminates the need for the user to hand carry a manager device. The manger device may include a display for displaying information to the user. Where the drug delivery system delivers insulin, the display may be used to display information such as blood glucose concentration readings, insulin on board and other information. The manager device may also include input elements, such as push buttons, knobs, keys or switches, for providing information, entering commands and/or initiating or halting activity.

The body conforming housing may be made of an elastomer that is arranged to be secured to the user. Electronic components and/or circuitry may be embedded in the elastomer. Alternatively, the electronic components or circuitry may be secured in a separate layer that is secured to the elastomer layer or may be sandwiched between two elastomer layers.

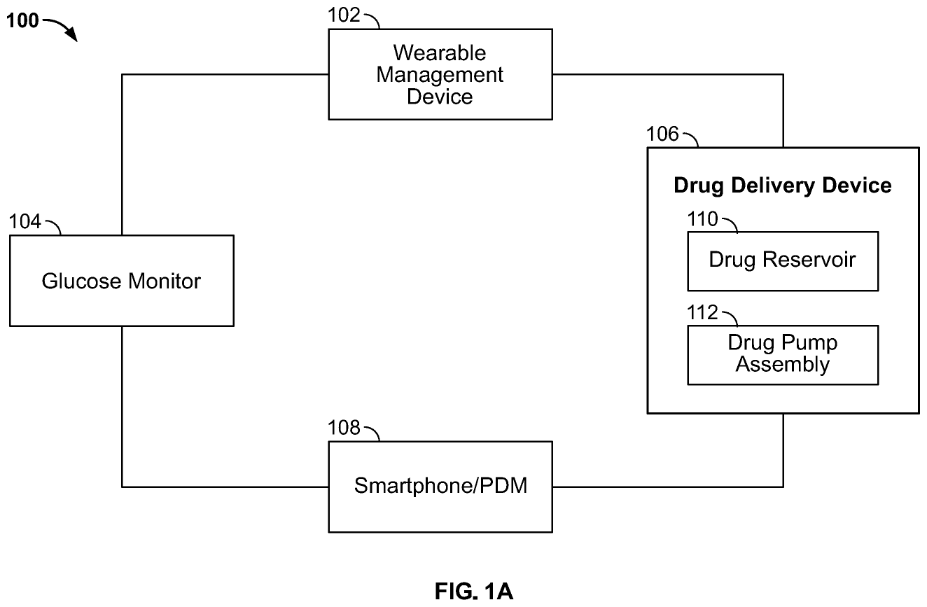

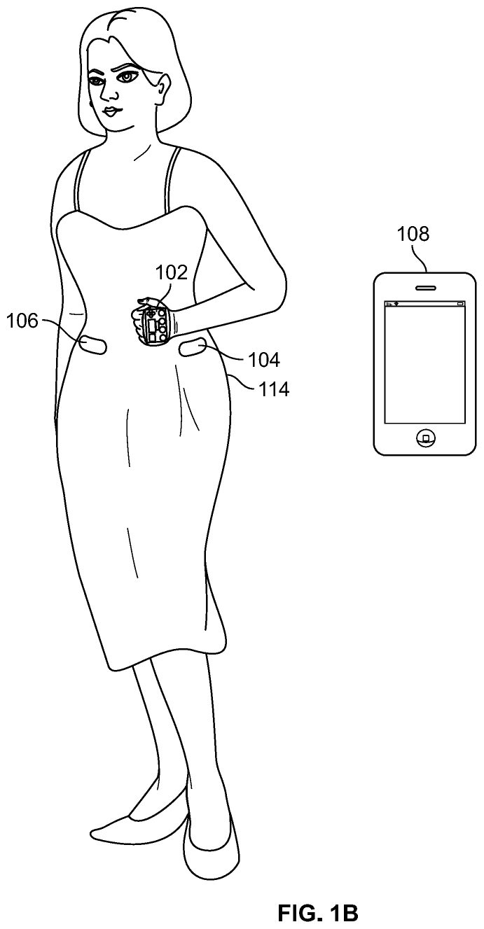

FIG. 1A depicts a high level block diagram of an illustrative drug delivery system 100 for an exemplary embodiment. A wearable management device 102 communicates with a glucose monitor (GM) 104, such as a continuous glucose monitor (CGM), and a drug delivery device 106. There may also be a smartphone/personal diabetes manager (PDM) 108. The smartphone/PDM 108 may be realized as a smartphone running software for providing management functionality, such as displaying information from the GM 104 and/or drug delivery device 106 and issuing commands to the drug delivery device 106 that may control drug delivery. In some instances, both the smartphone/PDM 108 and the wearable management device 102 may both be in use. In other instances, only the wearable management device 102 is in use. The functionality of the smartphone/PDM 108 and the wearable management device 102 may overlap partially or in whole.

The wearable management device 102 has a body conforming design to make it is easy be worn by a user. The wearable management device 102 may obtain and display information from the GM 104 and the drug delivery device 106. In addition, the wearable management device 102 may issue commands to the drug delivery device 106 to control activity of the drug delivery device 106.

The GM 104 may be affixed to a user and may provide blood glucose level readings. The readings may be, for instance, blood glucose concentration readings. Where the GM 104 is a CGM, the blood glucose readings may be provided on an ongoing basis.

The drug delivery device 106 may be, for example, a patch or a pump that is secured to the skin of a user. The drug delivery device 106 is responsible for delivering a drug to the user. The drug delivery device 106 may include a drug reservoir 110 for a drug to be delivered. The drug delivery device 106 may include a drug pump assembly 112 for pumping the drug out of the drug reservoir 110 to the user. The drug pump assembly 112 may include one or more needles or cannulas for delivering the drug to the user from the drug reservoir 110. In an exemplary embodiment, the drug delivery device 106 is an insulin pump that delivers insulin to a user. The drug delivery device 106 may deliver more than one drug to the user as needed. For instance, the drug delivery device 106 may deliver insulin and glucagon to a user as needed.

The wearable management device 102 may communicate with both the GM 104 and the drug delivery device 106. The communication may be over a communication line, such as via a wired connection, or may be over a wireless communication path, such as via the Bluetooth protocol, via Near Field Communication (NFC), a Wireless Body Area Network (WBAN) protocol, such as the IEEE 802.15.6 standard or via a wireless protocol such as the IEEE 802.11 wireless standard.

FIG. 1B shows an illustrative arrangement of the components of the drug delivery system 100 relative to a user 114. The drug delivery device 106 is secured to the abdomen of the user 114 via an adhesive. The GM 104 is also secured to the abdomen of the user 114. In the arrangement shown in FIG. 1B, the wearable management device 102 is secured to the wrist of the user 114. The smartphone/PDM 108 is not on body and must be held by hand to be used.

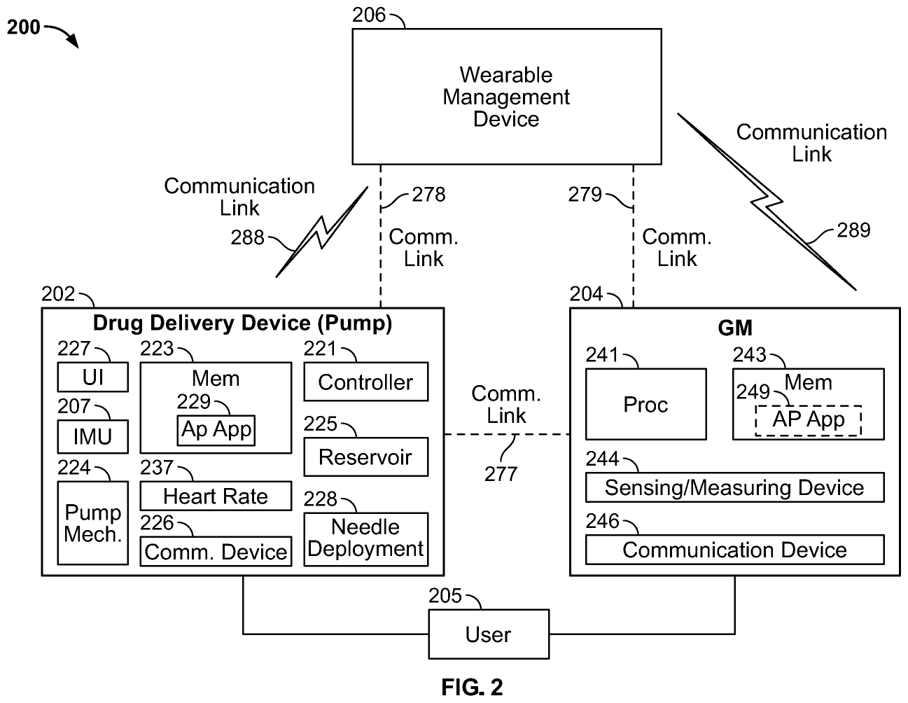

FIG. 2 depicts a more detailed view 200 of some of the components of the drug delivery device 202 and the GM 204. FIG. 2 illustrates the relationship of the drug delivery device 202 and the GM 204 with the wearable management device 206. The smartphone/PDM 108 is excluded from this figure as the wearable management device 206 is the focus.

As shown in FIG. 2, the drug delivery device 202 may include a pump mechanism 224 that may, in some examples, be referred to as a drug extraction mechanism or component, and a needle deployment mechanism 228. In various examples, the pump mechanism 224 may include a pump or a plunger. The needle deployment component 228 may, for example, include a needle, a cannula, and/or any other fluid path components for coupling the stored liquid drug in the reservoir 225 to the user 205. The needle or cannula may form a portion of the fluid path component coupling the user to the reservoir 225. After the needle deployment component 228 has been activated, a fluid path (not shown) to the user 205 is provided, and the pump mechanism 224 may expel the liquid drug from the reservoir 225 to deliver the liquid drug to the user via the fluid path. The fluid path may, for example, include tubing coupling the drug delivery device 202 to the user (e.g., tubing coupling the cannula to the reservoir 225).

The pump mechanism 224 may be communicatively coupled to the processor 221. The drug delivery device 202 may also include a power source (not shown), such as a battery, a piezoelectric device, or the like, for supplying electrical power to the pump mechanism 224 and/or other components (such as the controller 221, memory 223, and the communication device 226) of the drug delivery device 202. Although also not shown, an electrical power supply for supplying electrical power may similarly be included in each of the GM 204, the smart accessory device 207 and the smartphone/PDM 108.

A controller 221 is provided to oversee operation of the drug delivery device 202. The controller 221 may be implemented in hardware, software, or any combination thereof. The controller 221, for example, may be a microprocessor, a logic circuit, a field programmable gate array (FPGA), an application specific integrated circuit (ASIC) or a microcontroller coupled to a memory. The controller may execute computer program instructions. The controller 221 may maintain a date and time as well as other functions (e.g., calculations or the like) performed by processors. The controller 221 may be operable to provide artificial pancreas (AP) functionality to direct operation of the drug delivery device 202. The drug delivery system 200 may include components, such as IMU 207 and heart rate monitor 237 and functionality to determine a movement of the drug delivery device 202 that is indicative of physical activity of the user, implement an activity mode, a hyperglycemia mode, a hypoglycemia mode, and other functions, such as control of the drug delivery device 202. In addition, the controller 221 may be operable to receive data or information indicative of the activity of the user from the IMU 207, as well as from any other sensors (such as those (e.g., accelerometer, location services application or the like) on the management device 206 or CGM 204) of the drug delivery device 202 or any sensor coupled thereto, such as a global positioning system (GPS)-enabled device or the like.

The controller 221 may execute a control algorithm, such as the AP application 229, and other computer program instructions that may make the processor 221 operable to cause the pump to deliver doses of the drug or therapeutic agent to a user at predetermined intervals or as needed to bring BG measurement values to a target BG value. The size and/or timing of the doses may be programmed, for example, into the AP application 229 by the user or by a third party (such as a health care provider, drug delivery device manufacturer, or the like) using a wired or wireless link, such as 278 or 288, between the drug delivery device 202 and a management device 206 or other device, such as a computing device at a healthcare provider facility. The pump mechanism 224 of the drug delivery device 202 may be operable to receive an actuation signal, and in response to receiving the actuation signal and expel insulin from the reservoir 225 and the like.

The controller 221 may process the data from the IMU 207, heart rate monitor 237 or any other coupled sensor to determine if an alert or other communication is to be issued to the user and/or a caregiver of the user or if an operational mode of the drug delivery device 202 is to be adjusted. The controller 221 may provide the alert, for example, through the communications interface device 226. The communications interface device 226 may provide a communications link to one or more management devices physically separated from the drug delivery device 202 including, for example, the wearable management device 206 of the user and/or a caregiver of the user (e.g., a parent or HCP) or the smartphone/PDM (FIG. 1A), which is not shown in FIG. 2. The communication link provided by the communications interface device 226 may include any wired or wireless communication link operating according to any known communications protocol or standard, such as a Bluetooth protocol (including the Bluetooth Low Energy protocol), WiFi protocol (IEEE 802.11), WBAN protocol or a cellular standard.

The drug delivery device 202 may also include a user interface (UI) 227. The user interface 227 may include any mechanism for the user to input data to the drug delivery device 202, such as, for example, a button, a knob, a switch, a touch-screen display, or any other user interaction component. The user interface 227 may include any mechanism for the drug delivery device 202 to relay data to the user and may include, for example, a display, a touch-screen display, or any means for providing a visual, audible, or tactile (e.g., vibrational) output (e.g., as an alert). The user interface 227 may also include additional components not specifically shown in FIG. 2 for sake brevity and explanation. For example, the user interface 227 may include one or more user input or output components for receiving inputs from or providing outputs to a user or a caregiver (e.g., a parent or nurse), a display that outputs a visible alert, a speaker that outputs an audible, or a vibration device that outputs tactile indicators to alert a user or a caregiver of a potential activity mode, a power supply (e.g., a battery), and the like. Inputs to the user interface 227 may, for example, be a via a fingerprint sensor, a tactile input sensor, a button, a touch screen display, a switch, or the like. In yet another alternative, the activity mode of operation may be requested through the wearable management device 206 that is communicatively coupled to a controller 221 of the drug delivery device 202. In general, a user may generate instructions that may be stored as user preferences in a memory, such as 223 or 263 that specify when the system 200 is to enter the activity mode of operation.

The GM 204 may be physically separate from the drug delivery device 202 or may be an integrated component thereof. The GM 204 may provide the controller 221 with data indicative of measured or detected BG levels of the user. The GM 204 may be a device communicatively coupled to the controller 221 and may be operable to measure a BG value at a predetermined time interval, such as every 5 minutes, or the like. The GM 204 may provide a number of BG measurement values to the AP applications operating on the respective devices. For example, the GM 204 may be a continuous GM (CGM) that provides BG measurement values to the AP applications operating on the respective devices periodically, such as approximately every 5, 10, 12 minutes, or the like.

The GM 204 may also be coupled to the user 205 by, for example, adhesive or the like and may provide information or data on one or more medical conditions and/or physical attributes of the user. The information or data provided by the GM 204 may be used to adjust drug delivery operations of the drug delivery device 202. For example, the GM 204 may be a glucose sensor operable to measure BG and output a BG value or data that is representative of a BG value. For example, the GM 204 may be a glucose monitor that provides periodic BG measurements a CGM, or another type of device or sensor that provides BG measurements.

The GM 204 may include a processor 241, a memory 243, a sensing/measuring device 244, and communication device 246. The communication device 246 of GM 204 may include an electronic transmitter, receiver, and/or transceiver for communicating with the wearable management device 206 over a wireless link 279 or wired communication link 289. The GM may communicate with the delivery device 202 over wireless communication link 277. The sensing/measuring device 244 may include one or more sensing elements, such as a BG measurement element, a heart rate monitor, a blood oxygen sensor element, or the like. The processor 241 may include discrete, specialized logic and/or components, an application-specific integrated circuit, a microcontroller or processor that executes software instructions, firmware, programming instructions stored in memory (such as memory 243), or any combination thereof. For example, the memory 243 may store an instance of the AP application 249 that is executable by the processor 241.

Although the GM 204 is depicted as separate from the drug delivery device 202, in various examples, the GM 204 and drug delivery device 202 may be incorporated into the same unit. That is, in one or more examples, the GM 204 may be a part of the drug delivery device 202 and contained within the same housing of the drug delivery device 202 (e.g., the GM 204 may be positioned within or embedded within the drug delivery device 202). Glucose monitoring data (e.g., measured BG values) determined by the GM 204 may be provided to the drug delivery device 202, a smart accessory device (not shown) and/or the wearable management device 206, which may use the measured BG values to determine movement of the drug delivery device indicative of physical activity of the user, an activity mode, a hypoglycemia mode and a hyperglycemia mode.

The devices in the system 200, such as wearable management device 206 and GM 204, may also be operable to perform various functions including controlling the drug delivery device 202. The wearable management device 206 may control certain operations of the drug delivery device 202 and/or may be used to review data or other information indicative of an operational status of the drug delivery device 202 or a status of the user. The wearable management device 206 may receive alerts, notifications, or other communications from the drug delivery device 202 via one or more known wired or wireless communication standards or protocols.

Instructions for determining the delivery of the insulin (e.g., as a bolus dosage) to the user (e.g., the size and/or timing of any doses of the drug or therapeutic agent) may originate locally by the drug delivery device 202 or may originate remotely and be provided to the drug delivery device 202.

Although not shown, the system 200 may include a smart accessory device that may be, for example, an Apple Watch®, other smart device, including eyeglasses, provided by other manufacturers, a global positioning system-enabled, a fitness device, smart clothing, or the like. Similar to the management device 206, the smart accessory device (not shown) may also be operable to perform various functions including controlling the drug delivery device 202. For example, the smart accessory device may include a communication device, a processor, and a memory. The memory may store an instance of the AP application that includes programming code for providing the process examples described with reference to the examples described herein. The memory may also store programming code and be operable to store data related to the AP application.

The drug delivery device 202 may communicate with the GM 204 over a wireless link 277 and may communicate with the wearable management device 206 over a wireless link 288. The smart accessory device, when present, may communicate with the drug delivery device 202, the GM 204 and the wireless management device 206.

In various examples, the drug delivery system 200 may be an insulin drug delivery system. For example, the drug delivery device 202 may be the OmniPod® (Insulet Corporation, Acton, Mass.) insulin delivery device as described in U.S. Pat. Nos. 7,303,549, 7,137,964, or 6,740,059, each of which is incorporated herein by reference in its entirety or another type of insulin delivery device.

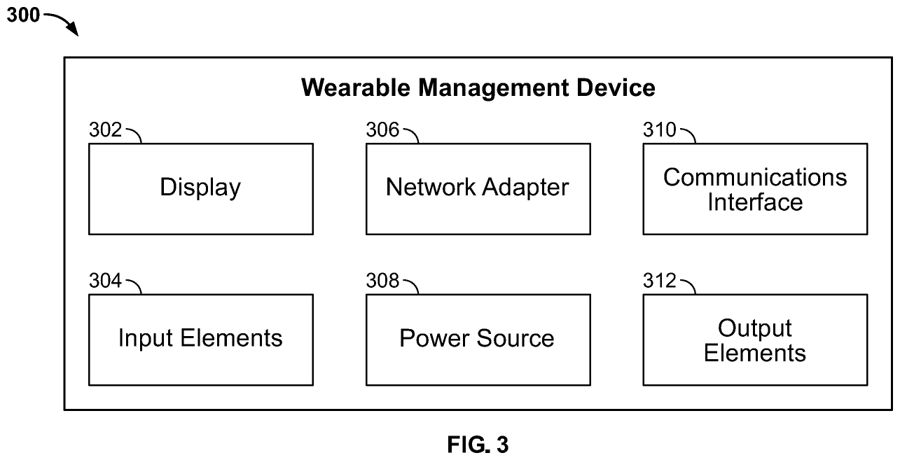

FIG. 3 depicts an illustrative block diagram of components that may be found in the wearable management device 300 in an exemplary embodiment. The wearable management device 300 may include a display 302, such as a Light Emitting Diode (LED) display, a Liquid Crystal Display (LCD) display, an electroluminescent (ELD) display or an e-paper display. In some embodiments, the display may be a touchscreen. The display 302 may be used to display information provided by the GM 104 and/or the drug delivery device 306. The wearable management device 300 may include input elements 304, such as buttons, switches or keys. In some instances, the wearable management device 300 may include a network adapter 306 for interfacing the wearable management device 300 with a Local Area Network (LAN), such as an 802.11 network, a BLE network, a WBAN network or a Bluetooth network. Alternatively, the network adapter 306 may connect the wearable management device 300 to a wired network, such as an Ethernet network. The network may provide access to a server or remote cloud computing resources.

A power source 308 may be provided for providing power to the wearable management device 200. The power source 308 may be a battery or may be electrical circuitry and a wire connection for connecting the wearable management device 300 to a remote device, like the drug delivery device 106 or the GM 104. Alternatively, the power source 308 may be an energy harvesting source that uses radio frequency, thermal, kinetic or triboelectric energy harvesting approaches. A communications interface 310 may be provided for facilitating communications, such as with the GM 104 and/or the drug delivery device 202. The communications interface 310 may include a transceiver for sending and receiving communications via a wired or wireless communication. The communications interface 310 may include a modem and may facilitate communications over a radio network like a cellular phone network, an 802.11 network, etc. or communications over a wired connection. Output elements 312 may also be provided on the wearable management device 300. The output elements 312 may include items such as an audio output component, like a loudspeaker, or visual output components, such as lights.

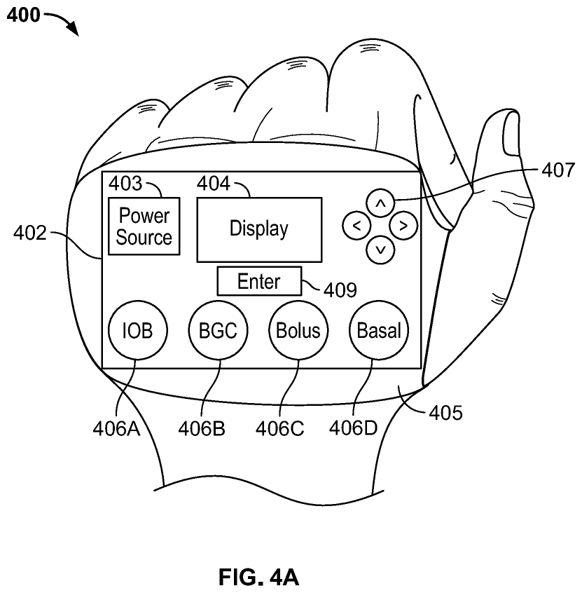

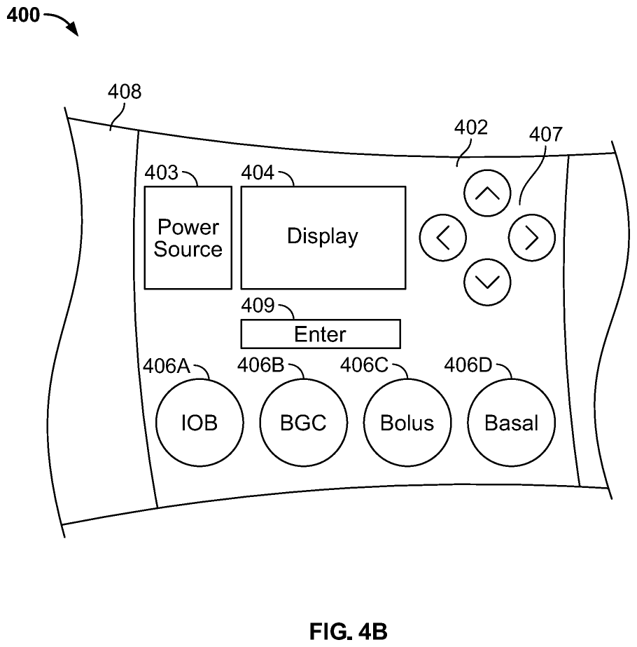

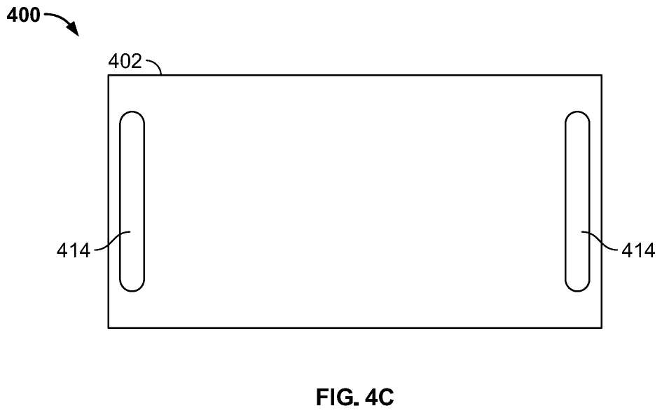

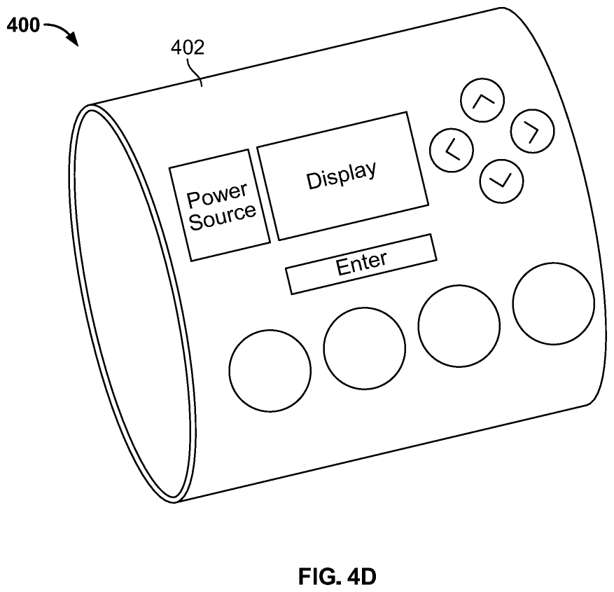

As was mentioned above, the wearable management device 300 may include a body conforming housing. FIG. 4A shows one example of a wearable management device 400 having a body conforming housing 402. In this instance, the body conforming housing fits around the hand 405 of the user. In FIG. 4A, the active components of the wearable management device 400 are positioned on the back of the hand 405 of the user. In this embodiment, the body conforming house may be like fingerless glove having openings for the fingers of the user to pass through. Alternatively, the housing 402 may be designed to adhere to the back of the user's hand 405 or may include straps, snaps, Velcro or other fastening means for securing the wearable management device 400 to the hand 405 of the user.

The wearable management device 400 may include a display 404 for displaying textual and graphical content. The wearable management device 400 also may include buttons 406A, 406B, 406C and 406D. Button 406A may be depressed by the user to display the IOB for the user on the display 404. Button 406B may be depressed by the user to display the blood glucose concentration (BGC) information, such as the most recent BGC reading or BGC history, on the display 404. Button 406C may be depressed by the user to request a bolus of insulin. Depressing the button 406C may cause a screen to be displayed on display 404 that allows a user to provide information needed for the bolus of insulin to be delivered. Button 406D may be depressed by the user to adjust basal insulin delivery or to view current basal settings on the display 404. Arrow buttons 407 may be provided to navigate a user interface provided on the display 404. An enter button 409 may be provided to enter information or select an item on the display 404.

A power source 403 for powering the wearable management device 400 is provided. As mentioned above, the power source 403 may be one or more batteries, such as coin cell batteries. The batteries may be disposable or rechargeable. The housing 402 may include a flap, door or other mechanism for gaining access to the batteries. A port may be built into the housing to facilitate recharging of the batteries. As was mentioned above, the power source 403 may include a solar component that is powered by light, such as sunlight. Other power harvesting mechanisms may be provided as part of the power source, such as ones that uses radio frequency, thermal, kinetic or triboelectric energy harvesting approaches.

As shown in FIG. 4B, the body-conforming housing 402 of the wearable management device 400 may be configured to fit to other portions of the body of the user. In FIG. 4B, the wearable management device 400 is secured to the wrist, arm or leg 408 of the user via the body-conforming housing 402. The body-conforming housing 402 may be a cylinder that is sized to fit around the wrist, arm or leg 408 of the user. FIG. 4C shows an example of an unrolled configuration 412 where the body-conforming housing resembles an unwound cylinder having fasteners 414, like Velcro, snaps, straps or the like. FIG. 4D shows the wearable management device 400 in a rolled configuration so that the body-conforming housing 402 forms a cylinder that is sized to wrap around the wrist, arm or leg of the user.

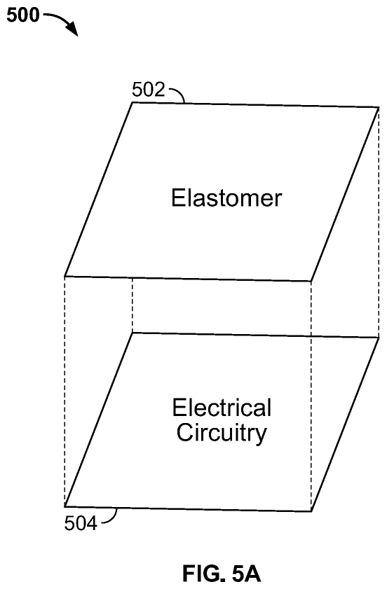

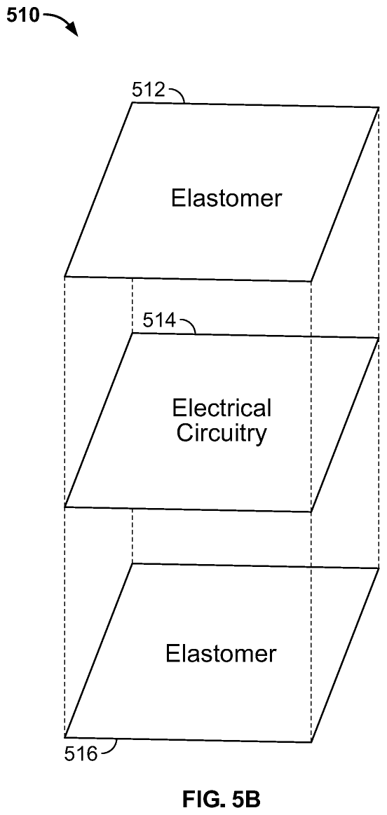

The body-conforming housing 402 may be formed of an elastomer in some exemplary embodiments. The advantages of an elastomer are that it is flexible, durable and readily conforms to a shape suitable for securing the wearable management device 400 to the user. In some embodiments, the wearable management device 500 may include an elastomer layer 502 that is coupled to or fused with a layer of electrical circuitry 504 as shown in FIG. 5A. The layer of electrical circuitry 504 may include the display 302, the input elements 304, the network adapter 306, the power source 308, the communications interface 310 and the output elements 312 of FIG. 3. In some embodiments, the layer 504 of electrical circuitry may be embedded into the elastomer layer 502. In other embodiments, additional layers (not shown) may be provided for protective reasons or for comfort of the user.

FIG. 5B shows another type of arrangement for the wearable management device 500 where the wearable management device includes an elastomer layer 502 and layer of electrical circuitry 504 as described above relative to FIG. 5A but also includes an additional elastomer layer 506. The layer of electrical circuitry 504 is sandwiched between the elastomer layers 502 and 506.

Those skilled in the art will appreciate that there may be other layers than those shown in FIGS. 5A and 5B in the wearable management device. For example, there may be additional layers not shown in FIGS. 5A and 5B.

Some of the input elements 304 may be formed as compressible button (like buttons 406A-406D, 407 or 409) or switches formed in the wearable management device by the elastomeric layer 502 in combination with the electrical circuitry. Structures may be provided to bias the buttons, switches, etc. to a non-contact state. For instance, ribs that force separation of the layers until compressed may be used. Use of a particularly small contact surface may also help facilitate bias toward a non-contact state. The height of the rib used, the ratio of rib to contact surface coverage, etc. can impact how easy/difficult it is to press the button, and as small of a contact surface as possible will minimize the impact of the bend radius. The electrical circuitry may include electrical contacts formed of a conductive metal, such as copper, gold or the like, that may be made to be in contact by compressing a structure formed in the housing, like a push button. A carbon-impregnated elastomer may be used in the elastomeric layer to facilitate the pushbutton.

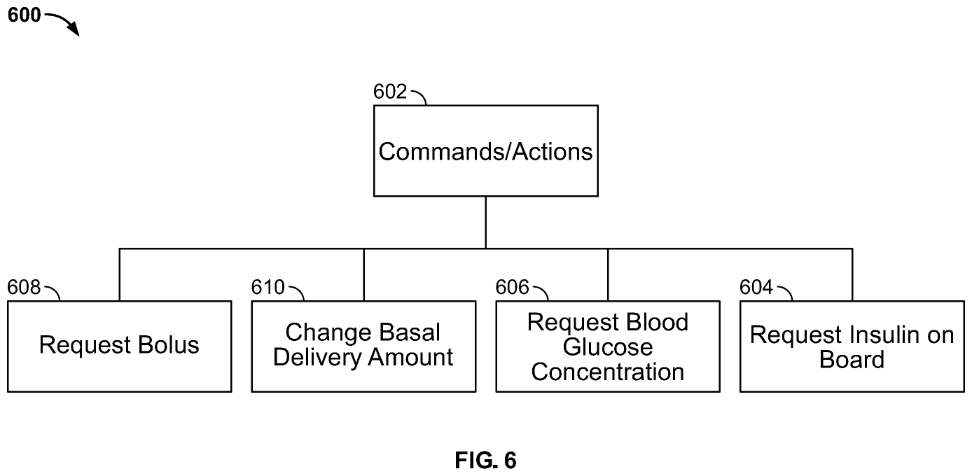

Using the input elements 304, a user may submit a number of commands or initiate actions as shown in the diagram 600 of FIG. 6. The listing of commands/actions 602 in FIG. 6 is meant to be illustrative and not exhaustive. Other commands/actions may be realized by way of the input elements 304 that differ from those depicted.

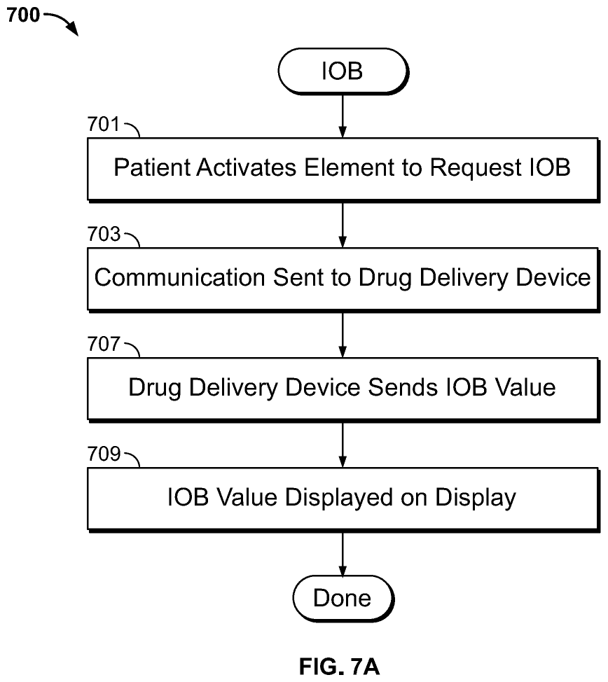

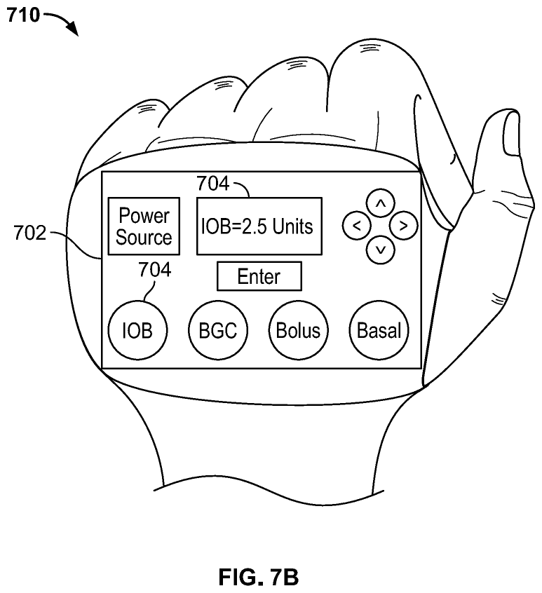

One possible command/action is to request the insulin on board (JOB). IOB refers to how much insulin is still active in the user of the body and may be calculated by the amount of insulin delivered to the user, the timing of the deliveries of insulin and the rate at which the insulin is consumed. FIG. 7A provides a flowchart 700 of the steps that may be taken to obtain the JOB. First, the user activates an input element to request the IOB (701). For example, as shown in FIG. 7B, the user may depress button 704 on the housing 702 of the wearable management device 710 for requesting the JOB. In response, a communication is sent from the wearable management device 102 (see FIG. 1A) via the communications interface 310 to the drug delivery device 106 (703). The drug delivery device 106 sends the IOB value back to the wearable management device 102 (707), where the IOB value (e.g., 2.5 units in FIG. 7B) may be displayed on the display 704 (709).

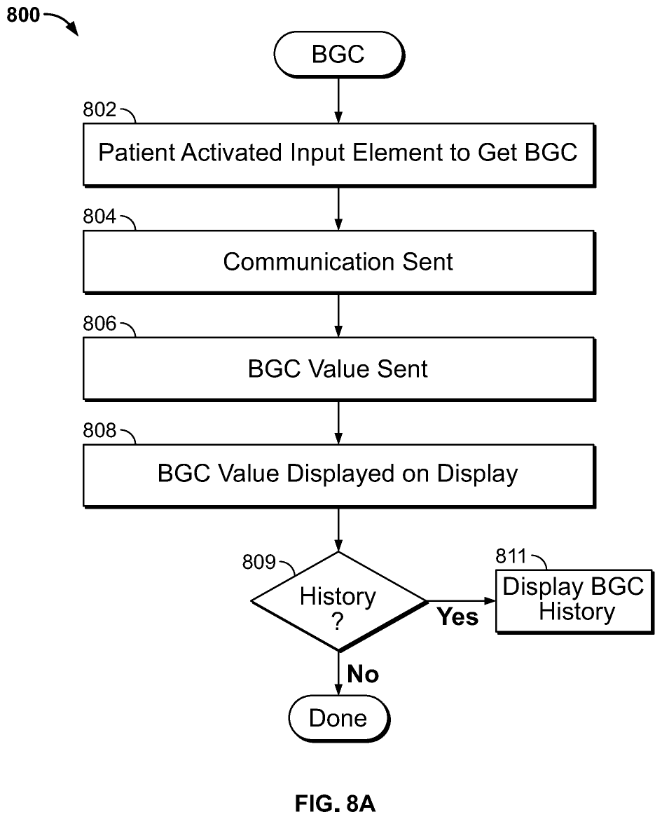

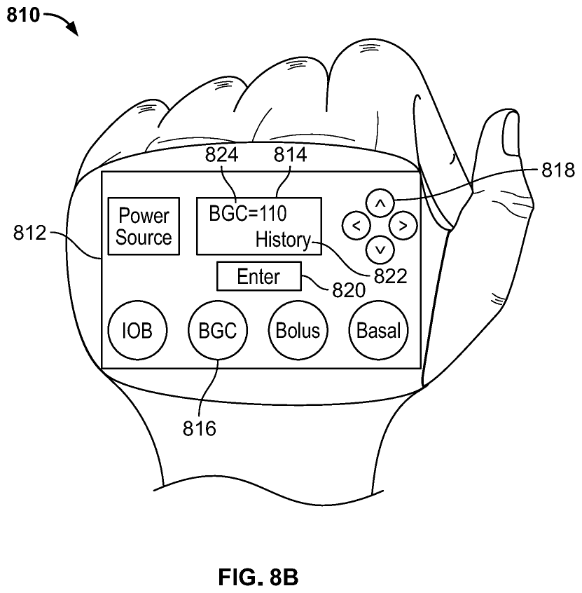

Another command/action is to request a blood glucose concentration (BGC) reading 606. FIG. 8A shows a flowchart 800 of illustrative steps that may be performed to obtain and display the BGC reading. The process may begin with the user depressing button 816 (see FIG. 8B) on the housing 812 of the wearable management device 810 to request the most recent BGC reading (802). In a first option, a communication may be sent via the communications interface from the wearable management device 102 to the GM 104 (804). In a second option, the drug delivery device 106 holds the BGC data and the communication is sent to the drug delivery device 106. The GM 104 or drug delivery device 106 reacts to the communication by sending the BGC reading to the wearable management device 102 (806), where the BGC reading 824 (e.g., 110 in FIG. 8B) is displayed on the display 814 (808).

A menu option 822 for seeing the BGC history may also be displayed on the display 814. The user may select this option 822 using the arrow keys 818 and the enter button 820 to cause the history to be displayed on the display 814 from the GM 104 (811). Thus, a decision (809) is made whether to display the CGC history or not.

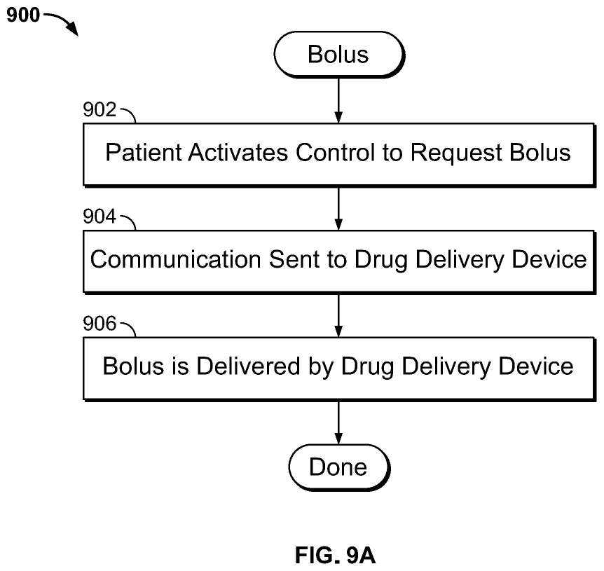

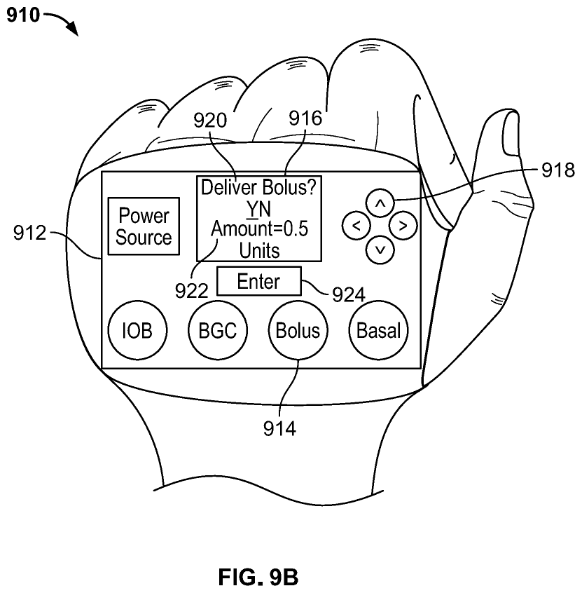

A further command/action is to request delivery of a bolus of drug (e.g., insulin) (610). FIG. 9A depicts a flowchart 900 of steps that may be performed re such a request. The user activates an input element, like button 914 (FIG. 9B) on the housing 912 of the wearable management device 910 to request the delivery of a bolus of the drug (902). As shown in FIG. 9B, the wearable management device 910 may request confirmation by a selection of Y or N (see 920 on display 916) using the arrow keys 918 and the enter button 924. In some instances, the user may specify an amount to be delivered, such as by selecting among options or by pressing up or down arrow buttons 918. As shown in FIG. 9B, the amount 922 may be shown, and the arrow keys 918 may be used to adjust the amount that is selected. Lastly, the amount may be selected by depressing the enter button 924. The request is sent from the wearable management device 102 to the drug delivery device 106 via the communications interface 310 (904). The bolus is then delivered to the user by the drug delivery device 106 (906). The drug delivery device 106 may have safety measures in place to ensure that the user is not over-bolusing by limiting the amount of bolus permitted based on IOB and other factors. Still further, the user may be required to press a sequence of buttons on the drug delivery device 106 to confirm basal or bolus drug deliveries. In some instances, the drug delivery device 106 may refuse the request.

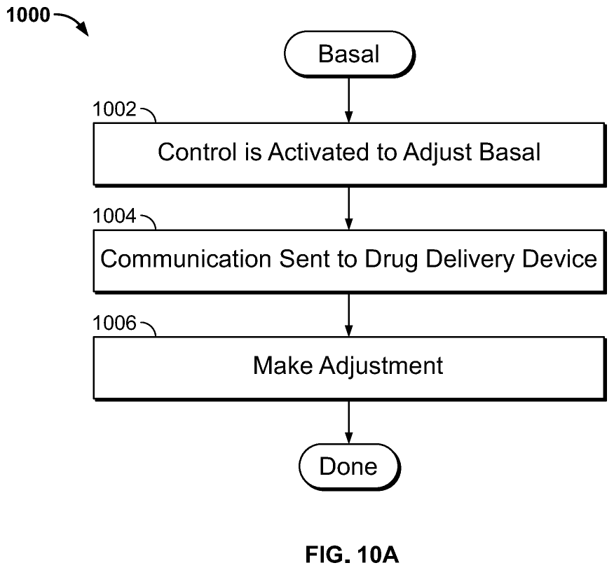

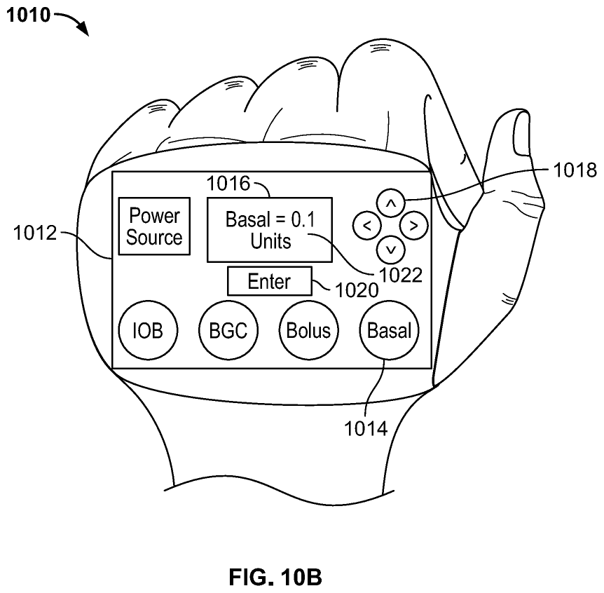

An additional illustrative command/action is to adjust a basal insulin delivery amount 610. FIG. 10A depicts a flowchart 1000 of illustrative steps relating to that command/action. An input element, such as button 1014 (FIG. 10B) on the housing 1012 of the wearable management device 1010, is activated to adjust the amount of basal insulin delivered (1002). The display 1016 may display the current basal amount obtained from the drug delivery device 106 or stored on the wearable management device 106. The current basal amount may be adjusted by arrow buttons 1018 after selecting the current amount 1022 on the display 1016. The basal amount may be limited to a maximum amount or a minimum amount. The revised amount may be chosen by depressing the enter button 1020. In response, a communication is sent via the communications interface 310 to the drug delivery device 106 (1004). The basal delivery amount is then adjusted by the drug delivery device 106 (1006). This may be a temporary adjustment, such as for a fixed period of time, or a permanent adjustment. The amount of insulin delivered may be modified as the adjustment. Another adjustment may be the relaxing or tightening of delivery constraints used in a control algorithm that controls delivery of the basal insulin.

While exemplary embodiments have been described herein, it should be appreciated that various changes in form and detail may be made without departing from the intended scope as defined in the appended claims.

1. A wearable device, comprising:
a user interface for displaying information to a user;
one or more activatable input elements formed in the wearable device for enabling the user to provide input to a drug delivery pump;
a communication interface for communicating with one or more external devices; and
a body conforming housing configured to make the wearable device by the user.

2. The wearable device of claim 1, wherein the body conforming housing of the wearable device includes at least one elastomeric layer.

3. The wearable device of claim 1, wherein the user interface comprises a display.

4. The wearable device of claim 1, wherein the user interface of the wearable device is configured to display user blood glucose information from a glucose monitor.

5. The wearable device of claim 1, wherein the communication interface enables communication with the glucose monitor.

6. The wearable device of claim 1, wherein the user interface of the wearable device is configured to display information regarding insulin on board.

7. The wearable device of claim 6, wherein the communication interface enables communication with the drug delivery pump.

8. The wearable device of claim 1, wherein the communication interface is a wireless communication interface.

9. The wearable device of claim 7, wherein the one or more activatable input elements formed in the wearable device is one of a button, a switch, a key, a wheel or a knob.

10. The wearable device of claim 9, wherein the activatable input elements includes an activatable input structure for requesting a delivery of a bolus of insulin to the user.

11. The wearable device of claim 1, wherein the wearable device is structured to be worn on at least one of an arm of the user, a wrist of the user or an arm of the user.

12. The wearable device of claim 1, wherein drug delivery pump is an insulin delivery pump.

13. A drug delivery system, comprising:
a drug delivery pump assembly, comprising:
a source of a drug to be delivered;
a pump for pumping the drug from the source;
a user interface for interfacing the pump with the user for delivery of the drug to the user; and
a drug delivery pump assembly housing for housing the drug delivery pump assembly and configured to be worn by the user; and
a wearable device, comprising:
a user interface for displaying information to the user;
one or more activatable input elements formed in the wearable device for enabling a user to provide input to the drug delivery pump assembly; and
a body conforming housing configured to make the wearable device by the user.

14. The drug delivery system of claim 13, wherein the drug is insulin and wherein the source of the drug is an insulin reservoir.

15. The drug delivery system of claim 13, wherein the body conforming housing of the wearable device includes at least one elastomeric layer.

16. The drug delivery system of claim 13, wherein the user interface of the wearable device is configured to display user blood glucose information.

17. The drug delivery system of claim 13, wherein the user interface of the wearable device is configured to display information regarding a quantity of insulin in the source.

18. The drug delivery system of claim 13, wherein the user interface of the wearable device is configured to display information regarding a quantity of insulin on board for the user.

19. The drug delivery system of claim 13, wherein the one or more activatable input elements formed in the wearable device is one of a button, a switch, a key, a wheel or a knob.

20. The drug delivery system of claim 13, wherein the wearable device is structured to be worn on at least one of an arm of the user, a wrist of the user or a hand of the user.
